(19) United States
(12) Patent Application Publication
(10) Pub. No.: US 2015/0284447 A1
(43) Pub. Date: Oct. 8, 2015

(54) METHOD OF ISOLATING SYNAGIS(R) IN THE ABSENCE OF BENZONASE

(71) Applicant: MEDIMMUNE, LLC, Gaithersburg, MD (US)

(72) Inventors: Min Wan, Gaithersburg, MD (US); Christopher Forespring, Gaithersburg, MD (US); Randall Lapcevich, Gaithersburg, MD (US); Erica Shane, Gaithersburg, MD (US); Cynthia Oliver, North Potomac, MD (US)

(21) Appl. No.: 14/440,640

(22) PCT Filed: Nov. 5, 2013
(86) PCT No.: PCT/US13/68403

§ 371 Date: May 5, 2015

Related U.S. Application Data
(60) Provisional application No. 61/722,590, filed on Nov. 5, 2012.

(51) Int. Cl. C07K 16/10; C07K 16/06

(52) U.S. Cl. CPC C07K 16/1027; C07K 16/065; C07K 2317/24; C07K 2317/565

(57) ABSTRACT

The present invention is directed to method of isolating an antibody from a composition. In some embodiments, the method comprises isolating Synagis® from a composition comprising Synagis®, the method comprising: (i) performing an ion exchange chromatography process on the composition; (ii) performing an affinity purification process on the composition; and (iii) performing a filtration process on the composition, wherein a final product comprising Synagis® results from (i), (ii), and (iii), wherein the final product is suitable for administration to a human and has a DNA concentration of <0.5 pg/mg, and wherein the method does not comprise adding benzonase to the composition.

This application contains a Sequence Listing electronically submitted via EFS-Web to the United States Patent and Trademark Office as an ASCII text filed entitled “RSVAB-300P l_SequenceListing_ST25.txt” having a size of 10 kilobytes and created on Nov. 4, 2013. The electronically submitted Sequence Listing serves as both the paper copy required by 37 CFR §1.821(c) and the CRF required by §1.821(e). The information contained in the Sequence Listing is incorporated by reference herein.

### FIELD OF THE INVENTION

invention is directed to method of isolating an antibody from a composition. In some embodiments, the method comprises isolating Synagis® (palivizumab) from a composition comprising Synagis®, the method comprising: (i) performing an ion exchange chromatography process on the composition; (ii) performing an affinity purification process on the composition; and (iii) performing a filtration process on the composition, wherein a final product comprising Synagis® results from (i), (ii), and (iii), wherein the final product is suitable for administration to a human and has a DNA concentration of ≦0.5 pg/mg, and wherein the method does not comprise adding benzonase to the composition.

### BACKGROUND OF THE INVENTION

Antibodies have been used in the treatment of various diseases and conditions and are generally derived from cell culture, using either eukaryotic or prokaryotic cell lines. However, antibodies used in pharmaceutical applications must have a high level of purity, especially in regard to contaminants from the cell culture, including cellular protein contaminants, cellular DNA contaminants, viruses and other transmissible agents. See “WHO Requirements for the use of animal cells as in vitro substrates for the production of biologicals: Requirements for Biological Substances No. 50.” No. 878. Annex 1, 1998.

In response to concerns about contaminants, The World Health Organization (WHO) established limits on the levels of various contaminants. For example, the WHO recommended a DNA limit of less than 10 ng per dose for protein products. Likewise, the United States Food and Drug Administration (FDA) set a DNA limit of less than or equal to 0.5 pg/mg protein.

To achieve the desired purity levels required for pharmaceutically acceptable antibodies, various methods for isolating the antibodies have been reported. These methods typically involve multiple steps and report clearance of host cell DNA in addition to other cell products and contaminants to reach a level of purity consistent with government regulation guidelines. The isolation steps vary depending on various factors, including the characteristics of the antibody being isolated, the quantity being produced, the expression system, and the growth media. However, generally the isolation process involves an initial lysis of the cells used to express the antibodies, a step for degrading DNA, one or more chromatography steps, a viral removal step, and a filtration step, just to name a few.

Processes to ensure the requisite purity can be expensive and time consuming. Therefore, the development of economically efficient purification methods is of increasing importance for the pharmaceutical and biotechnology industries.

### BRIEF SUMMARY OF THE INVENTION

The present invention is directed to method of isolating Synagis® from a composition. In some embodiments, the method comprises isolating Synagis® from a composition comprising Synagis®, the method comprising: (i) performing an ion exchange chromatography process on the composition; (ii) performing an affinity purification process on the composition; and (iii) performing a filtration process on the composition, wherein a final product comprising Synagis® results from (i), (ii), and (iii), wherein the final product is suitable for administration to a human and has a DNA concentration of ≦0.5 pg/mg, and wherein the method does not comprise adding benzonase to the composition.

In some embodiments, the method does not comprise adding an exogenous nuclease to the composition.

In some embodiments, the method of the invention further comprises conducting a virus inactivation process. For example, in some embodiments, the virus inactivation process comprises incubating the composition at a pH less than 4.0.

In some embodiments, the affinity purification process comprises a Protein A purification process. In some embodiments, the ion exchange chromatography process is a cation exchange chromatography process. In some embodiments, the cation exchange process comprises passing the antibody through a cationic resin selected from the group consisting of Capto S, S-Sepharose FF, and Poros 50 HS.

In some embodiments, the method further comprises a second ion exchange process. In some embodiments, the second ion exchange process is an anion exchange chromatography process.

In some embodiments, the anion exchange process comprises passing the antibody through an anionic membrane selected from the group consisting of Super Q, Natrix Q, Chromasorb Q and Mustang Q.

In some embodiments, the final product has an antibody yield of >80% (mol/mol). In some embodiments, the DNA concentration of the final product is ≦200 ng/mg.

In some embodiments, the composition is selected from the group consisting of serum of immunized animals, ascites fluid, hybridoma or myeloma supernatants, conditioned media derived from culturing a recombinant cell line, and cell extracts of immunoglobulin producing cells.

In some embodiments, the composition is from a bioreactor. In some embodiments, the composition has a volume greater than 100 liters. In some embodiments, the composition has a volume greater than 1000 liters.

In some embodiments, the affinity purification process occurs after the ion exchange process. In some embodiments, the filtration process occurs after the affinity process.

In some embodiments, the method comprises isolating Synagis® from a composition comprising Synagis®, the method comprising: (i) performing a cation exchange chromatography process on the composition to form a first product comprising the antibody; (ii) adding a buffer to the first product to form a buffered product; (iii) performing an affinity purification process on the buffered product to form a second product comprising the antibody; (iv) performing a filtration process on the second product to form a third product comprising the antibody; (v) performing a viral inactivation process on the third product; and (vi) formulating the third product to form a final product, wherein the final product comprising Synagis® is suitable for administration to a human and has a DNA concentration of ≦0.5 pg/mg; wherein the method does not comprise adding benzonase to the composition.

In some embodiments, the method comprises isolating Synagis® from a composition comprising Synagis®, the method comprising at least three of (i)-(v) listed below: (i) performing a cation exchange chromatography process on the composition; (ii) performing an affinity purification process on the composition; (iii) performing an ultrafiltration process on the composition; (iv) performing a viral inactivation process on the composition; and (v) performing an anion exchange chromatography process on the composition; wherein the product resulting from the at least three of (i)-(v) comprises Synagis®, is suitable for administration to a human and has a DNA concentration of ≦0.5 pg/mg; and wherein the method does not comprise adding benzonase to the composition.

### BRIEF DESCRIPTION OF THE FIGURES

FIG. 1 is a schematic representation of the both the “benzonase” and the “benzonase-free” process described in Example 1. The process includes a cationic exchange chromatography process, addition of a Tris/magnesium chloride buffer, a Protein A chromatography process, nanofiltration, low pH treatment, and an anion exchange chromatography process.

FIG. 2 is a schematic representation of the process described in Example 2. The left-hand column represents an isolation process wherein DNA is added, or “spiked” after the cation chromatography process. The right-hand column represents an isolation process wherein DNA is spiked after the low pH treatment process.

### DETAILED DESCRIPTION OF THE INVENTION

The present invention is based, in part, on the development of methods of isolating antibodies, or fragments thereof, in the absence of benzonase. In some embodiments, the method of the present invention provides for isolated antibodies, the method comprising: (i) performing an ion exchange chromatography process on the composition; (ii) performing an affinity purification process on the composition; and (iii) performing a filtration process on the composition, wherein a final product results from (i), (ii), and (iii), wherein the final product is suitable for administration to a human and has a DNA concentration of ≦0.5 pg/mg, and wherein the method does not comprise adding benzonase to the composition. In some embodiments, the antibody has an isoelectric point of greater than 8.0. In some embodiments, the antibody has an isoelectric point of greater than 9.0.

In some embodiments, the methods of the present invention enable a manufacturer to produce an antibody pharmaceutical product suitable for administration to a human in a more efficient manner, either by reducing costs, reducing method steps, reducing opportunities for error, reducing opportunities for introduction of unsafe or improper additives, etc., by omitting the addition of benzonase, or in some embodiments any exogenous nuclease. In the present invention, antibodies can be isolated without the addition of benzonase, which has been previously added during the isolation process of antibodies suitable for administration to a human. Benzonase refers to Benzonase® nuclease (Merck KGaA, United Kingdom), a genetically engineered 2 subunit (30 kDa each) endonuclease from Serratia marcescens, which degrades all forms for DNA and RNA (single-stranded, double-stranded, linear and circular). See, e.g., Benzonase Product sheet and U.S. Pat. No. 5,173,418, both of which are incorporated herewith in their entirety.

In some embodiments, the method of the present invention does not comprise adding an exogenous nuclease to the composition during the isolation process. Exogenous nuclease refers to the addition of any nuclease derived from, or originating, externally from the composition comprising the antibody being isolated. Thus, the term exogenous nuclease would include any nuclease added in protein form to the composition comprising the antibody. Exogenous nuclease would also include any nuclease expressed from genetic material derived from or originating externally from the composition, e.g., genetically modified organisms, wherein the genetic modification includes the insertion of genetic material encoding and capable of expressing a nuclease. In some embodiments, the methods described herein do not comprise adding an endonuclease. In some embodiments, the methods described herein do not comprise adding an exonuclease.

The methods described herein provide a process for isolating an antibody, e.g., Synagis®, from a composition, wherein the composition comprises the antibody and one or more impurities. The terms “isolate,” “isolating” and “isolation” refer to separating the antibody from an impurity or other contaminants in the composition. In some embodiments, at least 50%, 70%, 80%, 90%, 95%, 98%, 99%, 99.5%, or 99.9% (w/w) of an impurity is purified from the antibody. For example, in some embodiments, purification of an antibody, e.g. Synagis®, would comprise separating the antibody from 99% (w/w) of the host cell proteins present originally in the composition.

In some embodiments, the terms “isolate,” isolating” and “isolation” refer to separating an antibody, e.g. Synagis®, from an impurity or other contaminants in the composition to an extent consistent with guidelines of a governmental organization, e.g., the World Health Organization or the United States Food and Drug Administration. For example, “isolating” can refer to the removal of DNA from the composition to an extent wherein the final product comprises ≦0.5 pg DNA/mg protein.

The term “composition” as used herein refers to a mixture of an antibody, e.g., Synagis®, and one or more compounds, biologic material, and or any other molecules distinct from the antibody of interest. For purposes of convenience, all elements of the composition (e.g., compounds, biologic material, and or any other molecules distinct from the antibody of interest) other than the antibody of interest will be termed “impurities.” In some embodiments, the composition comprises a biologic, a cellular host organism (e.g., mammalian cells), and a growth media sufficient for propagating the host organism and allowing expression or production of the antibody. In some embodiments, the impurity can include a multimer (e.g., dimer, trimer, etc.) of the antibody of interest. In some embodiments, the impurity can include an undesired truncated form of the antibody, or an agglomerated form (e.g., misfolded or denatured form) of the antibody.

The term “composition” as used herein can undergo various transformations during the method of the present invention. For example, at the beginning of the method, the composition can comprise a relatively low concentration of antibody with high concentrations of impurities. As the method progresses, the concentration of one or more impurities may be reduced and/or the concentration of the antibody can be increased in the composition.

In some embodiments, the impurity can include an intact mammalian cell (e.g., Chinese hamster ovary cells (CHO cells) or murine myeloma cells (NSO cells)), or partial cells, e.g., cellular debris. In some embodiments, the impurity comprises a protein (e.g., soluble or insoluble proteins, or fragments of proteins, such as from host cell proteins), lipid (e.g., cell wall material), nucleic acid (e.g., chromosomal or extrachromosomal DNA), ribonucleic acid (t-RNA or mRNA), or combinations thereof, or any other cellular debris which is different from the antibody of interest. In some embodiments, the impurity can originate from the host organism that produced or contained the antibody of interest, e.g., Synagis®. For example, an impurity could be a cellular component of a prokaryotic or eukaryotic cell (e.g., cell wall, cellular proteins, DNA or RNA, etc.) that expressed a protein of interest. In some embodiments, the impurity is not from the host organism, e.g., an impurity could be from the cell culture media or growth media, a buffer, or a media additive. The impurity as used herein can include a single undesired component, or a combination of several undesired components.

In some embodiments, the composition is selected from the group consisting of serum of immunized animals, ascites fluid, hybridoma or myeloma supernatants, conditioned media derived from culturing a recombinant cell line, and cell extracts of immunoglobulin producing cells. The antibody of the present invention can be isolated from a composition comprising growth media and various eukaryotic cells, e.g., mammalian cells. One of skill in the art can select an appropriate cell line depending on the particulars of antibody of interest. The mammalian cells of the present invention, including the mammalian cells that are used in the methods of the invention, are any mammalian cells that are capable of growing in culture. Exemplary mammalian cells include, e.g., CHO, VERO, BHK, HeLa, CV1, MDCK, 293, 3T3, C127, PC12, HEK-293, PER C6, Sp2/0, NSO, W138 cells and myeloma cell lines (especially murine). Mammalian cells derived from any of the foregoing cells may also be used.

In some embodiments, the composition comprises a culturing medium, or concentrated cells originating from a culturing medium. The selection and use of culturing medium are known to those in the art. In some embodiments, the culturing medium is a cell culture media. Cell culturing media vary according to the type of cell culture being propagated. In some embodiments, the cell culturing media is a commercially available media. In some embodiments, the composition comprises a culturing medium which contains e.g., inorganic salts, carbohydrates (e.g., sugars such as glucose, galactose, maltose or fructose) amino acids, vitamins (e.g., B group vitamins (e.g., B12), vitamin A vitamin E, riboflavin, thiamine and biotin), fatty acids and lipids (e.g., cholesterol and steroids), proteins and peptides (e.g., albumin, transferrin, fibronectin and fetuin), serum (e.g., compositions comprising albumins, growth factors and growth inhibitors, such as, fetal bovine serum. newborn calf serum and horse serum), trace elements (e.g., zinc, copper, selenium and tricarboxylic acid intermediates) and combinations thereof. Examples of growth medium include, but are not limited to, basal media (e.g., MEM, DMEM, GMEM), complex media (RPMI 1640, Iscoves DMEM, Leibovitz L-15, Leibovitz L-15, TC 100), serum free media (e.g., CHO, Ham F10 and derivatives, Ham F12, DMEM/F12). Common buffers found in culturing media include PBS, Hanks BSS, Earles salts, DPBS, HBSS, and EBSS. Media for culturing mammalian cells are well known in the art and are available from, e.g., Sigma-Aldrich Corporation (St. Louis, Mo.), HyClone (Logan, Utah), Invitrogen Corporation (Carlsbad, Calif.), Cambrex Corporation (E. Rutherford, N.J.), JRH Biosciences (Lenexa, Kans.), Irvine Scientific (Santa Ana, Calif.), and others. Other components found in culturing media can include ascorbate, citrate, cysteine/cystine, glutamine, folic acid, glutathione, linoleic acid, linolenic acid, lipoic acid, oleic acid, palmitic acid, pyridoxal/pyridoxine, riboflavin, selenium, thiamine, transferrin. One of skill in the art will recognize that there are modifications to culturing media which would fall within the scope of this invention. In some embodiments, the culturing media can comprise a bovine product, e.g., serum albumin, transferrin, lipoprotein fraction, or combinations thereof. In some embodiments, the risk of transmitting bovine spongiform encephalopathy (BSE) is reduced by obtaining the bovine product from a source considered to be BSE-free by the United States Department of Agriculture and the European Community.

The term antibody refers to refers to polyclonal, monoclonal, multispecific, human, humanized or chimeric antibodies, single chain antibodies, Fab fragments, F(ab′)2 fragments, fragments produced by a Fab expression library, anti-idiotypic (anti-Id) antibodies, and epitope-binding fragments of any of the above. In some embodiments, the term “antibody” refers to a monoclonal antibody. The term “antibody” also refers to immunoglobulin molecules and immunologically active portions of immunoglobulin molecules, i.e., molecules that contain an antigen binding site that immunospecifically binds an antigen. The immunoglobulin molecules that can be purified by the method of the invention can be of any type (e.g., IgG, IgE, IgM, IgD, IgA and IgY), class (e.g., IgG1, IgG2, IgG3, and IgG4) or subclass of immunoglobulin molecule. In a preferred embodiment, the antibody is an IgG or IgA, most preferably an IgG.

In some embodiments, the antibody to be isolated is Synagis®. Synagis® (Synagis, MedImmune) is a recombinant humanized (chimeric murine-human) IgGlkappa monoclonal antibody glycoprotein with specificity for an epitope in the A antigenic site of the F (fusion) protein of respiratory syncytial virus (RSV). Palivizummab can be expressed from a stable murine (mouse) myeloma cell line (NS0). In some commercial embodiments, Synagis® is composed of two heavy chains (50.6 kDa each) and two light chains (27.6 kDa each), contains 1-2% carbohydrate by weight and has a molecular weight of 147.7 kDa Â±1 kDa (MALDI-TOF).

In some embodiments, a Synagis® antibody has a heavy chain having the amino acid sequence SEQ ID NO: 1 and a light chain having the amino acid sequence of SEQ ID NO: 6. In some embodiments, a Synagis® antibody includes the heavy chain variable region of the heavy chain amino acid sequence SEQ ID NO:1 or the heavy chain F_{AB} amino acid sequence SEQ ID NO: 2 and the light chain variable region of the light chain amino acid sequence SEQ ID NO:6. In some embodiments, a Synagis® antibody includes a heavy chain H1 complementarity determining region (CDR) having the amino acid sequence TSGMSVG (SEQ ID NO: 3), a heavy chain H2 CDR having the amino acid sequence DIWWDDKKDYNPSLKS (SEQ ID NO: 4), a heavy chain H3 CDR having the amino acid sequence SMITNWYFDV (SEQ ID NO: 5); a light chain L1 CDR having the amino acid sequence KCQLSVGYMH (SEQ ID NO: 7), a light chain L2 CDR having the amino acid sequence DTSKLAS (SEQ ID NO: 8), and a light chain L3 CDR having the amino acid sequence FQGSGYPFT (SEQ ID NO:9). The Synagis® antibody and its amino acid sequence are disclosed, for example, in Johnson et al., 1997, J. Infec. Dis; 76:1215-1224, and U.S. Pat. No. 5,824,307.

In some embodiments, the antibody to be isolated is a different commercially available antibody, selected from the group consisting of adalimumab (Humira®, Abbott Laboratories), eculizumab (Souris®, Alexion Pharmaceuticals), rituximab (Ritixan®, Roche/Biogen Idec/Chugai), infliximab (Remicade®, Johnson & Johnson/Schering-Plough/Tanabe), trastuzumab (Herceptin®, Roche/Chugai), bevacizumab (Avastin®, Chugai/Roche), palivizumab (Synagis®, MedImmune/Abbott), alemtuzumab (Campath®, Genzyme), and motavizumab (Numax®, MedImmune).

In some embodiments, the antibody to be isolated has an isoelectric point of greater than 8.0. In some embodiments, antibodies with high isoelectric points may tend to co-purify with acidic nucleic acids. Due to the propensity of DNA to copurify with the antibody of interested, and to eliminate trace amount of DNA in the final product, enzymatic digestion was traditionally utilized as a DNA reduction step. The inventors have found that the methods described here are sufficient to remove DNA in an antibody composition to a level consistent with governmental regulations, while omitting the use of benzonase, or in some embodiments any nuclease. In some embodiments, the antibody has an isoelectric point greater than 8.5, greater than 9.0, greater than 9.5, or greater than 10.0. In some embodiments, the antibody has an isoelectric point of 8.0-13.0, 8.5-12.0, 8.7-11.0, or 9.0-10.0. In some embodiments, the antibody has an isoelectric point of greater than about 9.0. In some embodiments, the antibody has an isoelectric point of greater than 9.0. Thus, in some embodiments, the method comprises isolating an antibody having an isoelectric point greater than 9.0 from a composition comprising the antibody, the method comprising: (i) performing an ion exchange chromatography process on the composition; (ii) performing an affinity purification process on the composition; and (iii) performing a filtration process on the composition, wherein a final product results from (i), (ii), and (iii), wherein the final product is suitable for administration to a human and has a DNA concentration of ≦0.5 pg/mg, and wherein the method does not comprise adding benzonase to the composition.

In some embodiments, antibodies other than Synagis® are isolated using the methods of the present inventions. Antibodies can also include chimeric, single chain, and humanized antibodies. Examples of antibodies can include commercialized antibodies, such as natalizmab (humanized anti-a4 integrin monoclonal antibody), humanized Anti-Alpha V Beta 6 monoclonal antibody, humanized anti-VLA1 IgG1 kappa monoclonal antibody; huB3F6 (humanized IgG1/kappa monoclonal antibody). In some embodiments, the antibody is a recombinant monoclonal antibody directed against CD-3, CD-4, CD-8, CD-19, CD-20, CD-34, CD-52, HER-4, HER-3, HER-2, TNF, and/or VLA-4. In some embodiments, the antibody is a recombinant monoclonal antibody directed against an epitope in the A antigenic site of the F protein of RSV.

An antibody produced by the method of the invention can be from any animal origin including birds and mammals. In some embodiments, the antibody purified by the methods of the invention are human, murine (e.g., mouse and rat), donkey, sheep, rabbit, goat, guinea pig, camel, horse, or chicken. As used herein, “human” antibodies include antibodies having the amino acid sequence of a human immunoglobulin and include antibodies isolated from human immunoglobulin libraries or from animals transgenic for one or more human immunoglobulin and that do not express endogenous immunoglobulins. See, e.g., U.S. Pat. No. 5,939,598 by Kucherlapati et al.

An antibody to be produced and used according to the invention can include, e.g., native antibodies, intact monoclonal antibodies, polyclonal antibodies, multispecific antibodies (e.g., bispecific antibodies) formed from at least two intact antibodies, antibody fragments (e.g., antibody fragments that bind to and/or recognize one or more antigens), humanized antibodies, human antibodies (Jakobovits et al., Proc. Natl. Acad. Sci. USA 90:2551 (1993); Jakobovits et al., Nature 362:255-258 (1993); Bruggermann et al., Year in Immunol. 7:33 (1993); U.S. Pat. Nos. 5,591,669 and 5,545,807), antibodies and antibody fragments isolated from antibody phage libraries (McCafferty et al., Nature 348:552-554 (1990); Clackson et al., Nature 352:624-628 (1991); Marks et al., J. Mol. Biol. 222:581-597 (1991); Marks et al., Bio/Technology 10:779-783 (1992); Waterhouse et al., Nucl. Acids Res. 21:2265-2266 (1993)). Anantibody purified by the method of the invention can be recombinantly fused to a heterologous polypeptide at the N- or C-terminus or chemically conjugated (including covalently and non-covalently conjugations) to polypeptides or other compositions. For example, an antibody purified by the method of the present invention can be recombinantly fused or conjugated to molecules useful as labels in detection assays and effector molecules such as heterologous polypeptides, drugs, or toxins. See, e.g., PCT publications WO 92/08495; WO 91/14438; WO 89/12624; U.S. Pat. No. 5,314,995; and EP 396,387.

According to the present invention, in some embodiments, the antibody can be produced or expressed by living cells, grown for example in a cell culture. The term “express” or “expression” as used herein refers to a process by which a gene produces a biochemical, for example, a polypeptide such as an antibody. The expression can include any manifestation of the functional presence of the gene within the cell including, without limitation, gene knockdown as well as both transient expression and stable expression. It can include without limitation transcription of the gene into messenger RNA (mRNA), and the translation of such mRNA into polypeptide(s). Thus, expression can include the creation of the antibody and any precursors. Expression of a gene can produce a “gene product,” wherein the gene product can be either a nucleic acid, e.g., a messenger RNA produced by transcription of a gene, or a polypeptide which is translated from a transcript. Gene products described herein further include nucleic acids with post transcriptional modifications, e.g., polyadenylation, or polypeptides with post translational modifications, e.g., methylation, glycosylation, the addition of lipids, association with other protein subunits, proteolytic cleavage, and the like. The antibody can also be produced by the cells, e.g. a metabolite produced by metabolic action of the cells, for example, a small molecule. The term “produced” includes both “expression” as described above and other methods in which a cell creates the biologic of interest.

Methods of isolating the antibody can include various means known in the art, e.g., centrifugation, size exclusion chromatography, ion exchange chromatography, affinity chromatography, filtration, and combinations of the above, just to name a few. The method of purification is generally chosen based on a characteristic of the antibody that distinguishes it from one or more impurities that coexist with the antibody in a composition. However, according to the methods provided herein, benzonase is not added or expressed at any point in the isolation process.

The methods as described herein can utilize an ion exchange chromatography process to isolate the antibody, e.g. Synagis®, from one or more impurities in the composition. Ion exchange chromatography refers to both cation exchange chromatography and anion exchange chromatography. For the purposes herein, “cation exchange chromatography” refers to any method by which a composition comprising the antibody and one or more impurities can be separated based on charge differences using a cation exchange matrix. A cation exchange matrix generally comprises covalently bound, negatively charged groups. Weak or strong cation exchange resins may be employed. Commonly, strong cation exchange resins comprise supported organic groups comprising sulphonic acid or sulphonate groups, depending upon the pH. Weak cation exchange resins commonly comprise supported organic groups comprising carboxylic acid or carboxylate groups, depending upon the pH. In certain embodiments, multimodal cation exchange resins can be used, which incorporate additional binding mechanisms as well as the ionic interactions, for example one or more of hydrogen bonding interactions and hydrophobic interactions. Examples of suitable cation exchange resins are well known in the art, and can include, but are not limited to Fractogel, carboxymethyl (CM), sulfoethyl (SE), sulfopropyl (SP), phosphate (P) and sulfonate (S), PROPAC WCX-10™ (Dionex), Capto S, S-Sepharose FF, Fractogel EMD SO₃M, Toyopearl Megacap II SP 550C, Poros 50 HS, and SP-sepharose matrix. In preferred embodiments, the cation resin is selected from Capto S, S-Sepharose FF, Fractogel EMD SO₃M, Toyopearl Megacap II SP 550C, Poros 50 HS, most preferably Poros 50 HS. In some embodiments, more than one cation exchange chromatography process can be employed on the composition. In some embodiments, the cation exchange chromatography process is employed in binding mode with respect to the antibody, i.e., is employed such that the antibody of interest is adsorbed to the cation exchange matrix, while one or more impurities are not adsorbed, thus isolating the antibody from the impurity. In some embodiments, the cation exchange matrix is washed one or more times with a buffer to remove additional impurities before the adsorbed antibody is removed from the cation exchange matrix. After one or more impurities have been removed from a composition employing cation exchange chromatography in binding mode, the adsorbed antibody can be eluted from the cation exchange matrix. Methods of eluting the antibody from the cation exchange are dependent on the matrix and are known to those of skill in the art.

Alternatively, in some embodiments the cation exchange process can be employed in flow-thru mode, i.e., is employed such that the antibody of interest is not adsorbed to the cation exchange matrix, while one or more impurities is adsorbed to the matrix, thus isolating the antibody from the impurity. In flow thru mode, one or more impurities are adsorbed to (or impeded by) the cation exchange matrix, and the antibody passes thru the matrix into the flow thru solution.

In some embodiments, the product of the cation exchange chromatography process, e.g., the eluate from a Poros 50 HS chromatography matrix, can result in a product having the characteristics in Table 1.

TABLE 1
Characteristics of product resulting from
cation exchange chromatography process

| Parameter | Preferred limit | | More preferred limit | |
|---|---|---|---|---|
| Product purity by HPSEC | ≧99% | | ≧99.5% | |
| DNA by PicoGreen | ≦500 | ng/mg | ≦10 | ng/mg |
| BSA level | ≦2,500 | ng/mg | ≦100 | ng/mg |
| Transferrin | ≦142 | ng/mg | ≦30 | ng/mg |

| | | |
|---|---|---|
| Reduced CGE-Light | 10.297-11.584 | 10.800-11.300 |
| chain migration time | minutes | |
| Reduced CGE-Heavy | 12.548-13.864 | 13.000-13.600 |
| chain migration time | minutes | minutes |
| Reduced CGE-Total | ≧90% | ≧98% |
| combined area of Product | | |
| Peaks | | |
| Reduced CGE-No Other | No other peak > 2% | No other peak > 1% |
| Peak | | |
| Process Step Yield | 70%-102% | 78%-95% |

In some embodiments, the ion exchange chromatography process is an anion exchange chromatography process. For the purposes herein, “anion exchange chromatography” refers to any method by which a composition comprising the antibody and one or more impurities can be separated based on charge differences using an anion exchange matrix. An anion exchange matrix generally comprises covalently bound, positively charged groups. Strong or weak anion exchange matrices can be employed. Examples of strong anion exchange matrices include, e.g., those having a quarternary ammonium ion. Examples of weak anion exchange matrices include, e.g., those having either a tertiary or secondary amine functional group, such as DEAE (diethylaminoethyl). In certain embodiments, multimodal anion exchange matrices can be used, which incorporate additional binding mechanisms as well as the ionic interactions, for example one or more of hydrogen bonding interactions and hydrophobic interactions. Examples of suitable anion exchange matrices are known in the art, and can include, but are not limited to Super Q, Sartobind Q, Natrix Q, Chromasorb Q, and Mustang Q. In some embodiments, the anion exchange matrix is Super Q. In some embodiments, more than one anion exchange process can be employed on the composition.

In some embodiments, the anion exchange chromatography process is employed in binding mode with respect to the antibody, i.e., is employed such that the antibody of interest is adsorbed to the anion exchange matrix, while one or more impurities do not bind, thus isolating the antibody from the impurity. In some embodiments, the anion exchange matrix is washed one or more times with a buffer to remove additional impurities before the adsorbed antibody is removed from the anion exchange matrix. After one or more impurities have been removed from a composition employing anion exchange chromatography in binding mode, the adsorbed antibody can be removed from the anion exchange matrix.

In some embodiments, the anion exchange process is employed in flow-thru mode, i.e., is employed such that the antibody of interest is not significantly adsorbed to the anion exchange matrix, while one or more impurities is adsorbed (or impeded) to the matrix, thus isolating the antibody from the impurity. After one or more impurities have been removed from a composition employing anion exchange chromatography in flow through mode, the adsorbed antibody can be obtained from the flow through of the anion exchange matrix.

In some embodiments, the method of the present invention can comprise more than one ion exchange process, e.g., a second ion exchange process. In some embodiments, the first ion exchange process is a cation exchange process and the second ion exchange process is an anion exchange process. In some embodiments, three ion exchange chromatography processes are used.

The methods described herein can utilize an affinity purification process to isolate the antibody from one or more impurities in the composition. As used herein, “affinity purification process” or “affinity chromatography” refers to a separation method whereby an antibody is purified by virtue of its specific binding properties to an affinity ligand for an antibody. In some embodiments, the functional affinity ligand can be immobilized on a solid or semi-solid support, so that when a composition comprising the antibody is passed over the ligand and the solid support, the antibody having a specific binding affinity to the ligand adsorbs to the ligand, and one or more other components of the composition are not adsorbed, or are bound at a lower affinity, and can be separated from the antibody. In some embodiments, the solid support comprising the ligand is washed one or more times with a buffer to remove additional impurities before the adsorbed antibody is removed from the ligand and the support. After one or more impurities have been removed, the adsorbed antibody can be removed from the ligand and the support, resulting in isolation of the antibody from the original composition.

Methods of removing the antibody from the ligand and support are dependent on the ligand and are known to those of skill in the art and can include, e.g., changes in environment, e.g., pH, addition of chaotropic agents or denaturants, or addition of commercially available elution buffers. In some embodiments, more than one affinity purification processes can be employed on the composition comprising the antibody.

Various affinity purification processes are know in the art, and include, but are not limited to, the use of Protein A, Protein G, or combinations thereof as ligands. The ligands can be immobilized on various supports, e.g., a resin. In some embodiments, the affinity purification process comprises a Protein A purification process, e.g., wherein the antibody is adsorbed to Protein A, and the Protein A is coupled to an immobilized support, e.g., a resin. Various Protein A affinity systems are available commercially, and include MabSelect, MabSelect SuRe, MabSelect Xtra, Sepaharose CL-4B, ProSep vA, ProSep vA Ultra, Ceramic HyperD, and Poros MabSelect. In some embodiments, the affinity purification process comprises a Protein G purification process, e.g., where the antibody is adsorbed to Protein G, and the protein G is couple to an immobilized support, e.g., a resin. Ready-to-use resins and purification kits are known to those in the art.

In some embodiments, the ligand is an antigen, e.g., a peptide or hapten, coupled to an immobilized support, wherein the antibody is selectively adsorbed to the antigen. Activated resins and complete kits for preparing immobilized antigens via a variety of chemistries are known to those in the art.

In some embodiments, other ligands can be used, and are known in the art. See, e.g., the reference texts Affinity Separations: A Practical Approach (Practical Approach Series), Paul Matejtschuk (Editor), Irl Pr (1997); and Affinity Chromatography, Herbert Schott, Marcel Dekker, New York (1997). For example, affinity ligands can include antibodies and antibody fragments, natural ligands or ligand analogs (e.g., for a particular receptor), and natural binding partners or analogues thereof (e.g., for a multisubunit complex).

In some embodiments, the composition undergoes multiple cycles of the affinity purification process. In some embodiments, the product of the affinity purification process, e.g., passage through a Protein A affinity matrix, can result in a product having the characteristics in Table 2:

TABLE 2
Characteristics of product resulting from affinity
purification process

| | Preferred | | More | |
|---|---|---|---|---|
| Parameter | limit | | preferred limit | |
| Product purity by HPSEC | ≧99% | | ≧99.5% | |
| Residual Protein A Level | ≦20 | ng/mg | ≦5 | ng/mg |
| BSA level | ≦12 | ng/mg | ≦5 | ng/mg |
| Transferrin | ≦4.7 | ng/mg | ≦2 | ng/mg |
| Host Cell Protein (NSO | ≦95 | ng/mg | ≦60 | ng/mg |
| ELISA) | | | | |
| Triton X-100 | ≦1.100 | ng/mg | ≦200 | ng/mg |

| | | |
|---|---|---|
| Reduced CGE- | 10.297-11.584 | 10.800-11.200 |
| Light Chain Migration | minutes | minutes |
| Time | | |
| Reduced CGE-Heavy | 12.548-13.864 | 13.100-13.600 |
| Chain Migration Time | minutes | minutes |
| Reduced CGE-Total | ≧90% | ≧98% |
| Combined Area of | | |
| Product Peaks | | |
| Reduced CGE-No Other | No other peak > 2% | No other peak > 1% |
| Peak | | |
| Process Step Yield | 84%-105% | 78%-95% |

The method of the present invention can utilizes a filtration process to isolate the antibody from one or more impurities in the composition. The terms “filtration process,” and “filtering” refer to the process of removing suspended particles from a composition by passing the composition through one or more semi-permeable filter (or membrane or medium) of a specified pore size diameter, wherein larger molecules (generally >10³-10⁶ Da) are retained on the filter, while water and lower molecular weight molecules pass through the filter.

In some embodiments, after filtration the antibody of the present invention is substantially in the permeate stream (i.e., it passes through the filter pores and is collected), while an impurity (e.g., cellular debris, DNA, and/or host cell protein) is substantially in the retentate stream. In some embodiments, after filtration the antibody of the present invention is substantially in the retentate stream, while an impurity is substantially in the permeate stream. The term “permeate stream” when referring to filtration, refers to the fraction of the composition that passes through the filter pores during filtration. The term “retentate stream” when referring to filtration, refers to the fraction of the composition that remains on the filter or that does not pass through the filter pores during filtration.

Suitable types of filtration apparatuses are known to those in the art and can be selected based on various factors, e.g., the molecular weight of the antibody to be filtered, the amount and size of the components of the composition to be filtered, the volume of the composition to be filtered, and the cell density and viability of the composition to be filtered. In some embodiments, filters, such as membrane ultrafilters, plate ultrafilters, cartridge ultrafilters, bag ultrafilters, or vacuum ultrafilters can be used. Commercially available ultrafilters that can be employed are manufactured by various vendors such as Millipore Corporation (Billerica, Mass.), Pall Corporation (East Hills, N.Y.), GE Healthcare Sciences (Piscataway, N.J.), and Sartorius Corporation (Goettingen, Germany).

In some embodiments, the method further comprises a virus inactivation process. As used herein, “virus inactivation process” refers to the (1) inactivation of a virus, (2) physical removal of a virus, or (3) combinations thereof. When referring to the inactivation of viruses, the viruses may remain in the final product, but in a non-infective form. In some embodiments, the virus inactivation process comprises incubating the composition, e.g., at a low pH sufficient to inactivate (e.g., denature) a virus. In some embodiments, the virus inactivation process comprises adjusting the pH of the composition to a pH of about 5.0 or less, about 4.5 or less, about 4.0 or less, or about 3.5 or less. In some embodiments, the pH of the composition is adjusted to a pH of about 1.0 to about 5.0, about 1.5 to about 4.5, about 2.0 to about 4.0, or about 2.5 to about 3.5. In some embodiments, the virus inactivation process comprises incubating the composition at a pH less than about 4.0, about 2.8 to about 3.2, or about 3.0. In some embodiments, the virus inactivation process comprises incubating the composition comprising the antibody at a pH of less than 4.0.

The pH of the composition can be lowered for various lengths of time sufficient for viral inactivation to occur, e.g., 1 minute to 2 hours, or 10 minutes to 90 minutes, preferably 20 minutes to 80 minutes, more preferably 25 minutes to 35 minutes, even more preferably about 30 minutes. Methods of altering pH are known to those of skill in the art.

In some embodiments, the viral inactivation process can include treatment with solvents or detergents, irradiation, and/or brief exposures to high temperatures sufficient to inactivate a virus. Methods of viral inactivation by these means are known to those of skill in the art, and one of skill in the art can select an appropriate treatment to be used during antibody isolation according to the present invention.

In some embodiments, the viral inactivation process can include the physical removal of the virus from the composition by means of nanofiltration. The term “nanofiltration” refers to the physical passing of the composition through a matrix, e.g., filter, membrane, etc., such that the antibody in the composition is separated from one or more viruses. In some embodiments, nanofiltration comprises passing the composition through a matrix having a pore size of less than 75 nm, 50 nm, 40 nm, 35 nm, 30 nm, 25 nm, 20 nm or 15 nm. Various nanofilters are available commercially and are known in the art.

In some embodiments, two separate virus inactivation processes are utilized, e.g., (1) a virus inactivation process comprising incubating the composition at a pH of less than 4.0, and (2) a virus inactivation process comprising a subjecting the composition to a nanofiltration process. In some embodiments, three or more separate virus removal processes are utilized.

In some embodiments, the methods described herein can result in a final product comprising the antibody, e.g. Synagis®, wherein the final product is suitable for administration to a human. As used herein, the term “suitable for administration to a human” includes a limit of less than 10 ng DNA/dose antibody product as established by the World Health Organization, “Requirements for the Use of Animal Cells as in vitro Substrates for the Production of Biologicals, (Requirements for Biological Substances No. 50),” World Health Organization, WHO Technical Report Series, No. 878, 1998). In some embodiments, the term “suitable for administration to a human” can include a more stringent limitation, depending on the antibody product. For example, in some embodiments, “suitable for administration to a human” can include less than 52.5 pg/dose, assuming a maximum dose of 105 mg protein based on the dosage of 15 mg antibody/kg body weight. In some embodiments, the term “suitable for administration to a human” can include less than or equal to 0.5 pg DNA/mg protein as determined by hybridization method. In some embodiments, the term “suitable for administration to a human” can include less than or equal to 0.4 pg DNA/mg protein, less than or equal to 0.3 pg DNA/mg protein, less than or equal to 0.2 pg DNA/mg protein, less than or equal to 0.1 pg DNA/mg protein as determined by hybridization method.

In some embodiments, the term “suitable for administration to a human” can include less than or equal to 25 ng DNA/mg protein as determined by PicoGreen method. In some embodiments, the term “suitable for administration to a human” can include less than or equal to 0.2 pg DNA/mg protein as determined by hybridization method.

The antibody isolation method described herein results in formation of a final product, wherein the final product is suitable for administration to a human and has a DNA concentration of ≦0.5 pg/mg. In some embodiments, the final product meets the parameters described in Table 3.

TABLE 3
Possible parameters of final product

| | | | More | |
|---|---|---|---|---|
| Parameter | Preferred Limit | | Preferred Limit | |
| Total Protein | 97-108 | mg/mL | 97-103 | mg/mL |
| Concentration | | | | |
| Product purity | Single product | | Single product | |
| by HPSEC | peak ≧ 99% | | peak ≧ 99.5% | |
| F-Protein | 80.2-120 | | 82-110 | |
| Binding | | | | |
| ELISA | | | | |
| Endotoxin | ≦5 EU/kg body | | ≦1 EU/kg body | |
| (LAL) | weight | | weight | |
| Bioburden | ≦2 CFU/10 mL | | ≦0.5 CFU/10 mL | |
| | weight | | | |
| Reduced CGE- | 10.3-11.6 minutes | | 10.4-11.3 | |
| Light chain | | | | |
| migration time | | | minutes | |
| Reduced CGE- | 12.5-13.9 minutes | | 12.6-13.7 | |
| Heavy | | | | |
| chain migration | | | minutes | |
| time | | | | |
| Reduced CGE- | ≧90% | | 98%-100% | |
| Total combined | | | | |
| area of Product | | | | |
| Peaks | | | | |
| Reduced CGE- | No other peak > 2% | | No other peak > 1% | |
| No Other Peak | | | | |
| NSO Host Cell | ≦90 | ng/mg protein | ≦5 | ng/mg protein |
| Protein | | | | |
| Bovine Serum | ≦9 | ng/mg protein | ≦5 | ng/mg protein |
| Albumin | | | | |
| Triton X-100 | ≦300 | ng/mg protein | ≦100 | ng/mg protein |

In some embodiments, final product meets the parameters described in Table 4:

TABLE 4
Additional possible parameters of final product

| Parameter | Preferred Limit | | More Preferred Limit | |
|---|---|---|---|---|
| Total Protein | 97-108 | mg/mL | 97-103 | mg/mL |
| Concentration | | | | |
| Insulin | ≦6 | μIU/mg protein | ≦2 | μIU/mg protein |
| Bovine Transferrin | ≦1.5 | ng/mg protein | ≦0.3 | ng/mg protein |
| Phenol Red | ≦6 | ng/mg protein | ≦3 | ng/mg protein |
| Bovine Lipoprotein | ≦816 | ng/mg protein | ≦210 | ng/mg protein |
| rProtein A ELISA | ≦20 | ng/mg protein | ≦2 | ng/mg protein |
| Benzonase | ≦0.0 | U/mg protein | ≦0.0 | U/mg protein |

The concentration of the antibody in the final product resulting from the method of the present invention can vary. In some embodiments, the method of the present invention results in a final product wherein the antibody is in a concentration of about 10 mg/ml to about 500 mg/mL, about 20 mg/mL to about 250 mg/mL, about 50 mg/mL to about 200 mg/mL, or about 75 mg/mL to about 150 mg/mL. In some embodiments, the method of the present invention results in a final product wherein the antibody is in a concentration of about 90 mg/ml to about 120 mg/mL, or about 100 mg/mL.

When isolating antibodies, in some embodiments large volumes of a composition can be present, e.g., during commercial manufacturing processes. Cell cultures expressing the antibodies to be isolated can be grown in a vessel appropriately sized for large-scale manufacture such as a bioreactor. Large volumes present several challenges for isolating processes. For example, the effect that a small change in flow rate through a filter has on the recovery of an isolated antibody is amplified when large volumes are used. Likewise, when using large volumes, the effect that a single step, process, or component is magnified, due to the scale of the step, process or component. For example, the economic cost of the addition of a single component in laboratory scale may be negligible, but the economic cost of addition of the same component in a large volume may be significant. The methods described herein can provide advantages for large volumes, since the omission of benzonase, or in some embodiments nuclease, can result in economic efficiencies not appreciated in laboratory-scale production. Thus, large volumes of a composition present unique problems that are amplified and have greater ramifications relative to the use of smaller volumes.

Thus, in some embodiments the present invention is directed to a method of isolating an antibody wherein the composition is from a bioreactor. In some embodiments, the composition is present in a large volume. The term “large volume” refers to volumes associated with the commercial and/or industrial production of an antibody. In some embodiments, the composition has a volume greater than 100 liters. In some embodiments, the composition has a volume greater than 1000 liters. In some embodiments, the composition has a volume of at least 500 liters, at least 750 liters, at least 1,000 liters, at least 1,250 liters, at least 1,500 liters, at least 2,000 liters, at least 5,000 liters or at least 10,000 liters.

In some embodiments, the method of the present invention provides for a step-wise isolation of the antibody using various ordered steps, resulting in formation of a final product. In some embodiments, the affinity exchange process is performed after the ion exchange process. In some embodiments, the filtration process is performed after the affinity purification process. In some embodiments, the ion exchange process is performed after affinity exchange process. In some embodiments, the affinity purification process is performed after the filtration process. In some embodiments, the method of isolating an antibody, e.g., Synagis®, from a composition comprising the antibody comprises: (i) performing a cation exchange chromatography process on the composition comprising the antibody to form a first product comprising the antibody; (ii) adding a buffer to the first product to form a buffered product; (iii) performing an affinity purification process on the buffered product to form a second product comprising the antibody; (iv) performing a filtration process on the second product to form a third product comprising the antibody; (v) performing a viral inactivation process on the third product; and (vi) formulating the third product to form a final product, wherein the final product comprising the antibody is suitable for administration to a human and has a DNA concentration of ≦0.5 pg/mg; wherein the method does not comprise adding benzonase to the composition.

In some embodiments, the method of isolating an antibody, e.g., Synagis®, from a composition comprising the antibody comprises at least three of (i)-(v) listed below: (i) performing a cation exchange chromatography process on the composition; (ii) performing an affinity purification process on the composition; (iii) performing an ultrafiltration process on the composition; (iv) performing a viral inactivation process on the composition; and (v) performing an anion exchange chromatography process on the composition; wherein the product resulting from the at least three of (i)-(v) is suitable for administration to a human and has a DNA concentration of ≦0.5 pg/mg; and wherein the method does not comprise adding benzonase to the composition.

The final product resulting from the methods as described herein can have an antibody yield of >80% (mol/mol). Antibody yield as described herein refers to the yield of the antibody in the final product relative to the amount of antibody present in the original composition before isolation of the antibody occurred. In some embodiments, the final product resulting from the methods as described herein can have an antibody yield of >85% (mol/mol), >90% (mol/mol), or >95% (mol/mol).

Various buffer systems can be used during the isolation process. In some embodiments, the buffer is selected from the group consisting of MES buffer, Tris buffer, sodium phosphate buffer, phthalate buffer, citrate buffer, acetate buffer and combinations thereof. In some embodiments, the buffer is a Tris buffer, preferably a Tris/magnesium buffer.

In some embodiments, the invention is directed to an antibody, e.g. Synagis®, made by any of the methods described herein.

In some embodiments, the antibody or composition comprising the antibody made by any of the methods described herein is pharmaceutically acceptable. “Pharmaceutically acceptable” refers to an antibody or composition that is, within the scope of sound medical judgment, suitable for contact with the tissues of human beings and animals without excessive toxicity or other complications commensurate with a reasonable benefit/risk ratio.

In some embodiments, the antibody isolated by the method of the present invention can be used in the treatment of a subject. As used herein, “subject” refers to any animal classified as a mammal, including humans and non-humans, such as, but not limited to, domestic and farm animals, zoo animals, sports animals, and pets. In some embodiments, subject refers to a human. While the invention is directed to method of isolating antibodies “suitable for administration to a human,” treatment using the isolated antibodies is not limited to solely human treatment.

The terms “treat” and “treatment” refer to both therapeutic treatment and prophylactic, maintenance, or preventative measures, wherein the object is to prevent or slow down (lessen) an undesired physiological condition, disorder or disease, or obtain beneficial or desired clinical results. For purposes of this invention, beneficial or desired clinical results include, but are not limited to, alleviation of symptoms or signs; diminishment of extent of condition, disorder or disease; stabilization (i.e., not worsening) of the state of condition, disorder or disease; delay in onset or slowing of condition, disorder or disease progression; amelioration of the condition, disorder or disease state, remission (whether partial or total), whether detectable or undetectable; or enhancement or improvement of condition, disorder or disease. Treatment includes eliciting a clinically significant response, without excessive levels of side effects. For example, in some embodiments where the antibody is Synagis®, treatment can refer to the prevention of, reduction of occurence of, treatment of, reduction of, or alleviation of symptoms associated with infection of respiratory syncytial virus.

In some embodiments, the antibody or final product comprising the antibody made by the method described herein is administered to a subject in a therapeutically effective amount. The term “therapeutically effective amount” refers to an amount of antibody that diminishes one or more symptoms of a disease or disorder (i.e., treats a disease or disorder) in a subject. In some embodiments, the term “therapeutically effective amount” refers to an amount of antibody sufficient to achieve a desired physiologic state. The precise therapeutic dosage of an antibody necessary to be therapeutically effective can vary between subjects (e.g., due to age, body weight, condition of the subject, the nature and severity of the disorder or disease to be treated, and the like). In some embodiments, the term “therapeutically effective amount” refers to an amount of antibody sufficient to achieve a desired physiological state. In some embodiments, the therapeutically effective amount cannot be specified in advance and can be determined by a caregiver, for example, by a physician or other healthcare provider, using various means, for example, dose titration. Appropriate therapeutically effective amounts can also be determined by routine experimentation using, for example, animal models.

The route of administration of the isolated antibody product of the method of the present invention can be via, for example, oral, parenteral, inhalation or topical modes of administration. The term parenteral as used herein includes, e.g., intravenous, intraarterial, intraperitoneal, intramuscular, subcutaneous, rectal or vaginal administration. In some embodiments, the isolated antibody is Synagis® and the route of administration is intramuscular injection. While all these forms of administration are clearly contemplated as being within the scope of the invention, a preferred form for administration would be a solution for injection, in particular for intravenous or intraarterial injection or drip. Usually, a suitable pharmaceutical composition for injection may comprise a buffer (e.g. acetate, phosphate or citrate buffer), a surfactant (e.g. polysorbate), optionally a stabilizer agent (e.g. human albumin), etc.

The pharmaceutical compositions containing the antibody made by the method of the invention can comprise pharmaceutically acceptable carriers, including, e.g., ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, polyethylene-polyoxypropylene-block polymers, and polyethylene glycol. In some embodiments, the methods described herein provide a method of isolating Synagis®, the method comprising: (i) performing a cation exchange chromatography process on the composition to form a first product comprising the antibody; (ii) adding a buffer to the first product to form a buffered product; (iii) performing an affinity purification process on the buffered product to form a second product comprising the antibody; (iv) performing a filtration process on the second product to form a third product comprising the antibody; (v) performing a viral inactivation process on the third product; and (vi) formulating the third product to form a final product, wherein the final product comprises Synagis® and is suitable for administration to a human and has a DNA concentration of ≦0.5 pg/mg; wherein the method does not comprise adding benzonase to the composition.

The present invention includes a method of isolating Synagis® from a composition comprising Synagis®, the method including:
i. performing an ion exchange chromatography process on the composition;
ii. performing an affinity purification process on the composition; and
iii. performing a filtration process on the composition;
   wherein a final product comprising Synagis® results from (i), (ii), and (iii), wherein the final product is suitable for administration to a human and has a DNA concentration of ≦0.5 pg/mg, and wherein the method does not comprise adding benzonase to the composition.

The present invention includes a method of isolating Synagis® from a composition having Synagis®, the method including:
i. performing a cation exchange chromatography process on the composition to form a first product comprising Synagis®;
ii. adding a buffer to the first product to form a buffered product;
ii. performing an affinity purification process on the buffered product to form a second product comprising Synagis®;
iv. performing a filtration process on the second product to form a third product comprising Synagis®;
v. performing a viral inactivation process on the third product; and
vi. formulating the third product to form a final product comprising Synagis®,
   wherein the final product is suitable for administration to a human and has a DNA concentration of ≦0.5 pg/mg;
   wherein the method does not comprise adding benzonase to the composition.

The present invention includes a method of isolating Synagis® from a composition having Synagis®, the method including at least three of (i)-(v):
i. performing a cation exchange chromatography process on the composition;
ii. performing an affinity purification process on the composition;
iii. performing an ultrafiltration process on the composition;
iv. performing a viral inactivation process on the composition; and
v. performing an anion exchange chromatography process on the composition;
   wherein the product resulting from the at least three of (i)-(v) comprises Synagis® and is suitable for administration to a human and has a DNA concentration of ≦0.5 pg/mg; and
   wherein the method does not comprise adding benzonase to the composition.

In some embodiments of the methods of the present invention, the method does not include adding an exogenous nuclease to the composition.

In some embodiments of the methods of the present invention, the method further includes a virus inactivation process. In some aspects, the virus inactivation process includes incubating the composition at a pH less than 4.0.

In some embodiments of the methods of the present invention, the antibody is an IgG.

In some embodiments of the methods of the present invention, the affinity purification process includes a Protein A purification process.

In some embodiments of the methods of the present invention, the ion exchange chromatography process is a cation exchange chromatography process. In some embodiments, the cation exchange process includes adsorbing the antibody to a cationic resin selected from Capto S, S-Sepharose FF, and/or Poros 50 HS.

In some embodiments of the methods of the present invention, the method, further includes a second ion exchange process. In some embodiments, the second ion exchange process is an anion exchange chromatography process. In some embodiments, the anion exchange process includes passing the antibody through an anionic membrane selected from Super Q, Natrix Q, Chromasorb Q and/or Mustang Q.

In some embodiments of the methods of the present invention, the final product has an antibody yield of about >80% (mol/mol).

In some embodiments of the methods of the present invention, the DNA concentration of the final product is about ≦200 ng/mg.

In some embodiments of the methods of the present invention, the composition is serum of immunized animals, ascites fluid, hybridoma or myeloma supernatants, conditioned media derived from culturing a recombinant cell line, and/or cell extracts of immunoglobulin producing cells.

In some embodiments of the methods of the present invention, the composition includes a preparation from a bioreactor.

In some embodiments of the methods of the present invention, the composition has a volume greater than about 100 liters.

In some embodiments of the methods of the present invention, the composition has a volume greater than about 1000 liters.

In some embodiments of the methods of the present invention, the process of (ii) occurs after the process of (i).

In some embodiments of the methods of the present invention, the process of (iii) occurs after the process of (ii).

In some embodiments of the methods of the present invention, the Synagis® antibody includes a heavy chain having the amino acid sequence SEQ ID NO: 1 and a light chain having the amino acid sequence of SEQ ID NO: 6.

In some embodiments of the methods of the present invention, the Synagis® antibody includes the heavy chain variable region of SEQ ID NO:1 or SEQ ID NO: 2 and the light chain variable region of the light chain SEQ ID NO:6.

In some embodiments of the methods of the present invention, the Synagis® antibody includes a H1 complementarity determining region (CDR) having the amino acid sequence TSGMSVG (SEQ ID NO: 3), a H2 CDR having the amino acid sequence DIWWDDKKDYNPSLKS (SEQ ID NO: 4), a H3 CDR having the amino acid sequence SMITNWYFDV (SEQ ID NO: 5); a L1 CDR having the amino acid sequence KCQLSVGYMH (SEQ ID NO: 7), a L2 CDR having the amino acid sequence DTSKLAS (SEQ ID NO: 8), and a L3 CDR having the amino acid sequence FQGSGYPFT (SEQ ID NO:9).

Throughout the present disclosure, all expressions of percentage, ratio, and the like are “by weight” unless otherwise indicated. As used herein, “by weight” is synonymous with the term “by mass,” and indicates that a ratio or percentage defined herein is done according to weight rather than volume, thickness, or some other measure.

As used herein, the term “about,” when used in conjunction with a percentage or other numerical amount, means plus or minus 10% of that percentage or other numerical amount. For example, the term “about 80%,” would encompass 80% plus or minus 8%.

### EXAMPLES

### Example 1

### Synagis® Isolation with and without Addition of Benzonase

### 1. Preparation of Cell Culture Supernatant

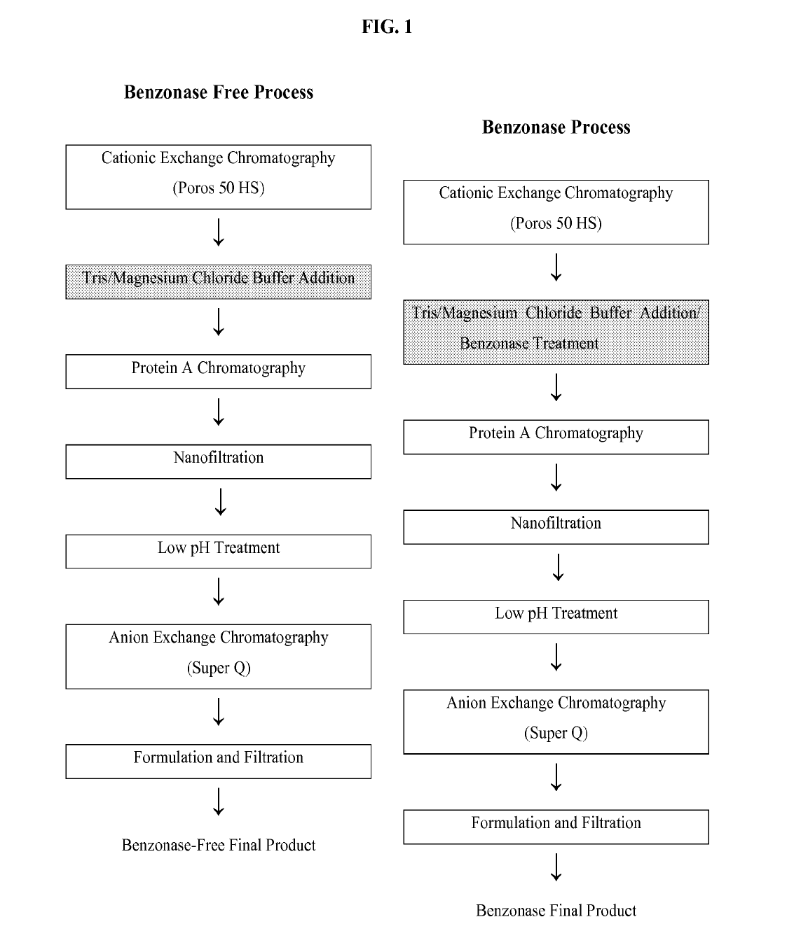

Synagis®, a humanized monoclonal IgG₁ antibodies targeting respiratory syncytial virus (RSV) protein F, was expressed in NS0 cells using serum-free DMNSO-4 medium and harvested from a production bioreactor on two separate runs, termed “Benzonase” run and “Benzonase-free” run. The NS0 cells and cellular debris from each run were removed by centrifugation and filtration. The resultant clarified harvest materials for each run was pH and conductivity adjusted to achieve a load pH of 6.0 and a conductivity of 6.0 mS/cm, and the process stream proceeded directly through a Pod-like depth filter. The Synagis® was isolated from the harvested cellular composition as described in steps 2-8 below, and as outlined in FIG. 1.

### 2. Cation Exchange Chromatography

The harvest material of both the “benzonase” and the “benzonase-free” runs of step 1 were loaded onto separate Poros 50 HS column (180 cm), washed, and eluted from the column. The loading, washing, and elution process was controlled for dynamic binding capacity (load: ≦20 g/L resin/cycle) and volumetric flowrate (linear flowrate: 330 cm/hr), and was completed in multiple cycles to accommodate the entire volume. The output parameters of the product of the Poros 50 HS column are defined in Table 5:

TABLE 5
Output parameters for product of Poros 50 HS Chromatography

| | Acceptance | Benzonase | | | Benzonase-free | | |
|---|---|---|---|---|---|---|---|
| Parameter | Criteria | Lot 1 | Lot 2 | Lot 3 | Lot 4 | Lot 5 | Lot 6 |
| Product purity by | ≧99% | 100% | 100% | 100% | 100% | 100% | 99% |
| HPSEC | | | | | | | |
| DNA by PicoGreen | ≦500 ng/mg | 3 ng/mg | 3 ng/mg | 3 ng/mg | 3 ng/mg | 4 ng/mg | 3 ng/mg |
| BSA level | ≦2,500 ng/mg | 73 | 106 | 156 | 88 | 67 | 95 |
| Transferrin | ≦142 ng/mg | 3 | 24 | 9 | 3 | 4 | 3 |
| Reduced CGE - Light | 10.297-11.584 minutes | 11.261 | 11.010 | 10.995 | 10.879 | 10.849 | 10.839 |
| chain migration time | | | | | | | |
| Reduced CGE - | 12.548-13.864 minutes | 13.594 | 13.268 | 13.276 | 13.164 | 13.151 | 13.157 |
| Heavy chain | | | | | | | |
| migration time | | | | | | | |
| Reduced CGE - Total | ≧90% | 99% | 99% | 99% | 99% | 99% | 99% |
| combined area of | | | | | | | |
| Product Peaks | | | | | | | |
| Reduced CGE - No | No other | 1 | 1 | 1 | 1 | 1 | 1 |
| Other Peak | peak >2% | | | | | | |
| Process Step Yield | 70%-102% | 93 | 85 | 84 | 80 | 84 | 83 |

### 3. Tris/Magnesium Chloride (TM) Buffer Addition

The pH of the cation exchange chromatography product from both the “benzonase” and the “benzonase-free” runs from step 2 was adjusted to 8.5±0.2 using 1 M Tris base solution. The pH of the product pool after the addition of Tris base solution was 8.5. TM buffer (20 mM Tris, 102 mM MgCl₂, pH 8.5) was added to the pH-adjusted pool to achieve a final MgCl₂ concentration of 2±0.2 mM (0.018 L/L to 0.022 L/L). To the “benzonase” fractions, 12,500 Units of benzonase was added, and incubated for 18-48 hrs. No benzonase was added to the “benzonase-free” fractions. Both the “benzonase” and the “benzonase-free” fractions were subjected to Protein A affinity chromatography.

### 4. Protein A Affinity Chromatography

The buffered fractions from both the “benzonase” and the “benzonase-free” runs from step 3 was loaded onto a Protein A column (rProtein A Sepharose® Fast Flow resin; 140 cm), washed, and then eluted from the column. The loading, washing, and eluting process was completed in five cycles to accommodate the entire volume of buffered product. The process was controlled for dynamic binding capacity (load: ≦18 g/L resin/cycle) and volumetric flowrate (35±4 L/min) The eluted product from each run was collected in two different tanks. The eluted product had the following characteristics as described in Table 6.

TABLE 6
Output parameters for product of Protein A Chromatography

| | | Acceptance | Benzonase | | | Benzonase-free | | |
|---|---|---|---|---|---|---|---|---|
| Parameter | | Criteria | Lot 1 | Lot 2 | Lot 3 | Lot 4 | Lot 5 | Lot 6 |
| Product purity | Tank 1 | ≧99% | 100% | 100% | 100% | 100% | 100% | 100% |
| by HPSEC | Tank 2 | | 100% | 100% | 100% | 100% | 100% | 100% |
| Protein | Tank 1 | ≦13.0 g/L | 6.6 | 6.2 | 6.2 | 7.1 | 5.9 | 6.7 |
| Concentration | Tank 2 | | 7.1 | 6.3 | 6.3 | 7.1 | 6.2 | 7.3 |
| Residual | Tank 1 | ≦20 ng/mg | 2 | 2 | 2 | 2 | 2 | 1 |
| Protein A | Tank 2 | | 1 | 2 | 2 | 2 | 1 | 1 |
| Level | | | | | | | | |
| BSA level | Tank 1 | ≦12 ng/mg | 3 | 2 | 1 | 2 | 1 | 3 |
| | Tank 2 | | 2 | 2 | 2 | 3 | 1 | 3 |
| Transferrin | Tank 1 | ≦4.7 ng/mg | <1.0 | <1.0 | <1.0 | <1.4 | <1.7 | <1.5 |
| | Tank 2 | | <0.7 | <1.0 | <1.0 | <1.4 | <1.6 | <1.4 |
| Host Cell | Tank 1 | ≦95 ng/mg | <48 | <52 | <52 | <34 | <54 | <36 |
| Protein (NSO | Tank 2 | | <45 | <51 | <52 | <34 | <52 | <33 |
| ELISA) | | | | | | | | |
| Triton X-100 | Tank 1 | ≦1,100 ng/mg | <152 | <161 | <161 | <141 | <169 | <149 |
| | Tank 2 | | <141 | <159 | <159 | <141 | <161 | <137 |
| Reduced CGE - | Tank 1 | 10.297-11.584 minutes | 10.986 | 11.013 | 11.039 | 10.881 | 11.115 | 10.928 |
| Light Chain | Tank 2 | | 11.270 | 11.257 | 10.983 | 10.882 | 10.827 | 10.928 |
| Migration Time | | | | | | | | |
| Reduced CGE- | Tank 1 | 2.548-13.864 minutes | 13.266 | 13.277 | 13.391 | 13.159 | 13.470 | 13.228 |
| Heavy Chain | Tank 2 | | 13.606 | 13.616 | 13.238 | 13.161 | 13.126 | 13.227 |
| Migration Time | | | | | | | | |
| Reduced CGE - | Tank 1 | 90% | 99% | 99% | 99% | 99% | 99% | 99% |
| Total | Tank 2 | | 99% | 99% | 99% | 99% | 99% | 99% |
| Combined Area | | | | | | | | |
| of Product | | | | | | | | |
| Peaks | | | | | | | | |
| Reduced CGE - | Tank 1 | No other | 1 | 1 | 1 | 1 | 1 | 1 |
| No Other | Tank 2 | peak >2% | 1 | 1 | 1 | 1 | 1 | 1 |
| Peak | | | | | | | | |
| Process Step | Tank 1 | 84%-105% | 93 | 85 | 84 | 80 | 84 | 83 |
| Yield | ank 2 | | | | | | | |

### 5. Nanofiltration

The product of both the “benzonase” and the “benzonase-free” runs from the Protein A affinity chromatography from step 4 was filtered using an Asahi Kasei Planova 15N hollow-filter cartridge (scale: 8-10×4 m²; transmembrane pressure: <13 psi; load: ≦800 g/m³ filter area). After nanofiltration, the filter was flushed with equilibration buffer to maximize recovery. Nanofilter integrity was confirmed following use. The nanofiltered product was filtered into a storage tank through 0.2 μm membrane filters for storage at room temperature. Yield was 94-99%, except for one lot of the “benzonase-free” run, which had a lower yield due to inadvertent mishandling of the product sample.

### 6. Low pH Treatment

Following nanofiltration, the product stream from of both the “benzonase” and the “benzonase-free” runs from step 5 was titrated to a pH 3±0.1 using a glycine solution and was incubated for approximately 30 minutes at room temperature. The product was then neutralized to pH of 7.6±0.4.

### 7. Anion Exchange (Super Q) Chromatography

The low pH-treated product from both the “benzonase” and the “benzonase-free” runs step 6 was passed though a Super Q 650M resin column (Scale: 140 cm). The process was controlled for column load pH, conductivity, dynamic binding capacity (load: ≦79 g/L resin/cycle) and volumetric flowrate (linear flowrate ≦330 cm/hr). Yields for each lot ranged from 95-99%. Contaminant DNA levels of the product of anion exchange chromatography were determined by hybridization method as presented in Table 7.

TABLE 7
Output parameters of Super Q Chromatography

| | Acceptance | Benzonase | | | Benzonase-free | | |
|---|---|---|---|---|---|---|---|
| Parameter | Criteria | Lot 1 | Lot 2 | Lot 3 | Lot 4 | Lot 5 | Lot 6 |
| DNA by | ≦0.5 pg/mg | <0.1 | <0.1 | <0.0 | <0.1 | <0.1 | <0.1 |
| hybridization | | | | | | | |

### 8. Ultrafiltration/Diafiltration

Following anion exchange chromatography, the product of both the “benzonase” and the “benzonase-free” runs from of step 7 was concentrated by ultrafiltration/diafiltration (scale: 450-600 ft²; load: ≦60 g/ft² filter area) into a formulation buffer using a minimum of 5 buffer exchanges. After diafiltration was complete, the ultrafiltration system was flushed with formulation buffer to maximize recovery. The product concentration was adjusted to a target of 103±6 g Synagis®/L with formulation butter. The product pH was about 6 and final permeate conductivity about 1.1 mS/cm. Yield of each lot ranged from 100% to 103%. The resulting final product was passed through a −/2 μm filter and stored at 2° C. to 8° C.

### Example 2

### Synagis® Isolation with and without Addition of Benzonase

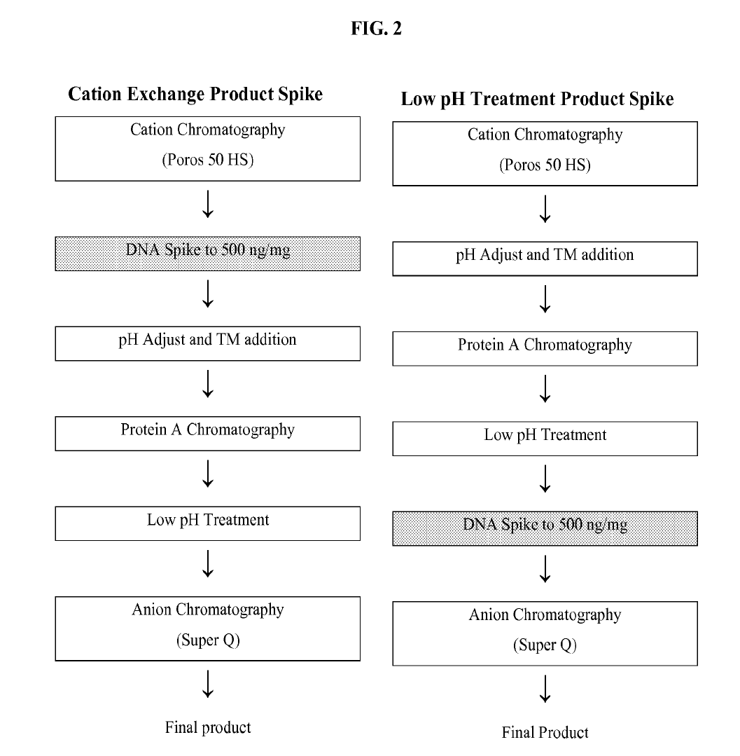

The efficiency and robustness of the methods described herein for isolating an antibody from a composition spiked with an excess of exogenous DNA was investigated. A antibody from a benzonase-free composition was produced, harvested and isolated as described in Example 1, except that exogenous DNA was added at two process steps. 500 ng/mg mouse genomic DNA (Novagen) was added to the benzonase-free sample either (i) after the cationic chromatography process (Example 1, step 2), or (ii) after the low pH treatment process (Example 1, step 6). Except for the addition of the excess of exogenous DNA, the antibody isolation then proceeded as described in Example 1. Elution volume, elution titer and step yield were monitored throughout the isolation processes and were consistent with manufacturing trends. A schematic of the experiment is presented in FIG. 2. The DNA concentration of both “spiked” samples was determined by the hybridization method and NSO PCR method. The DNA concentration was <0.1 pg/mg (hybridization method) and <0.044 pg/mg (NSO PCR method) for the sample spiked after the cationic chromatography process, and <0.3 pg/mg (hybridization method) and <0.041 pg/mg (NSO PCR method) for the sample spiked after the low pH treatment. This result indicates that the methods described herein are sufficiently efficient and robust to remove DNA, even when additional amounts of DNA is added to the composition.

All of the various embodiments or options described herein can be combined in any and all variations. While the invention has been particularly shown and described with reference to some embodiments thereof, it will be understood by those skilled in the art that they have been presented by way of example only, and not limitation, and various changes in form and details can be made therein without departing from the spirit and scope of the invention. Thus, the breadth and scope of the present invention should not be limited by any of the above described exemplary embodiments, but should be defined only in accordance with the following claims and their equivalents.

All documents cited herein, including journal articles or abstracts, published or corresponding U.S. or foreign patent applications, issued or foreign patents, or any other documents, are each entirely incorporated by reference herein, including all data, tables, figures, and text presented in the cited documents.

What is claimed is:

1. A method of isolating Synagis® from a composition comprising Synagis®, the method comprising:
i. performing an ion exchange chromatography process on the composition;
ii. performing an affinity purification process on the composition; and
iii. performing a filtration process on the composition;
wherein a final product comprising Synagis® results from (i), (ii), and (iii), wherein the final product is suitable for administration to a human and has a DNA concentration of ≦0.5 pg/mg, and wherein the method does not comprise adding benzonase to the composition.

2. A method of isolating Synagis® from a composition comprising Synagis®, the method comprising:
i. performing a cation exchange chromatography process on the composition to form a first product comprising Synagis®;
ii. adding a buffer to the first product to form a buffered product;
iii. performing an affinity purification process on the buffered product to form a second product comprising Synagis®;
iv. performing a filtration process on the second product to form a third product comprising Synagis®;
v. performing a viral inactivation process on the third product; and
vi. formulating the third product to form a final product comprising Synagis®, wherein the final product is suitable for administration to a human and has a DNA concentration of ≦0.5 pg/mg;
wherein the method does not comprise adding benzonase to the composition.

3. A method of isolating Synagis® from a composition comprising Synagis®, the method comprising at least three of (i)-(v):
i. performing a cation exchange chromatography process on the composition;
ii. performing an affinity purification process on the composition;
iii. performing an ultrafiltration process on the composition;
iv. performing a viral inactivation process on the composition; and
v. performing an anion exchange chromatography process on the composition;
wherein the product resulting from the at least three of (i)-(v) comprises Synagis® and is suitable for administration to a human and has a DNA concentration of ≦0.5 pg/mg; and
wherein the method does not comprise adding benzonase to the composition.

4. The method of claim 1, wherein the method does not comprise adding an exogenous nuclease to the composition.

5. The method of claim 1, further comprising a virus inactivation process.

6. The method of claim 5, wherein the virus inactivation process comprises incubating the composition at a pH less than 4.0.

7. The method of claim 1, wherein the antibody is an IgG.

8. The method of claim 1, wherein the affinity purification process comprises a Protein A purification process.

9. The method of claim 1, wherein the ion exchange chromatography process is a cation exchange chromatography process.

10. The method of claim 9, wherein the cation exchange process comprises adsorbing the antibody to a cationic resin selected from the group consisting Capto S, S-Sepharose FF, and Poros 50 HS.

11. The method of claim 1, further comprising a second ion exchange process.

12. The method of claim 11, wherein the second ion exchange process is an anion exchange chromatography process.

13. The method of claim 12, wherein the anion exchange process comprises passing the antibody through an anionic membrane selected from the group consisting of Super Q, Natrix Q, Chromasorb Q and Mustang Q.

14. The method of claim 1, wherein the final product has an antibody yield of >80% (mol/mol) and/or wherein the DNA concentration of the final product is ≦200 ng/mg.

15. The method of claim 1, wherein the composition is selected from the group consisting of serum of immunized animals, ascites fluid, hybridoma or myeloma supernatants, conditioned media derived from culturing a recombinant cell line, cell extracts of immunoglobulin producing cells, and a bioreactor preparation.

16. The method of claim 1, wherein the composition has a volume greater than 100 liters.

17. The method of claim 1, wherein the composition has a volume greater than 1000 liters.

18. The method of claim 1, wherein the process of (ii) occurs after the process of (i).

19. The method of claim 1, wherein the process of (iii) occurs after the process of (ii).

20. The method of claim 1 wherein Synagis® comprises:
a heavy chain having the amino acid sequence SEQ ID NO: 1 and a light chain having the amino acid sequence of SEQ ID NO: 6;
a heavy chain variable region of SEQ ID NO:1 or SEQ ID NO: 2 and a light chain variable region of the light chain SEQ ID NO:6; or
a H1 complementarity determining region (CDR) having the amino acid sequence TSGMSVG (SEQ ID NO: 3), a H2 CDR having the amino acid sequence DIWWDDKKDYNPSLKS (SEQ ID NO: 4), a H3 CDR having the amino acid sequence SMITNWYFDV (SEQ ID NO: 5); a L1 CDR having the amino acid sequence KCQLSVGYMH (SEQ ID NO: 7), a L2 CDR having the amino acid sequence DTSKLAS (SEQ ID NO: 8), and a L3 CDR having the amino acid sequence FQGSGYPFT (SEQ ID NO:9).


SEQUENCE LISTING (9 sequences)

SEQ ID NO: 1 - protein, 450 aa, Artificial sequence
  Heavy chain of SYNAGIS humanized antibody
QVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 227 aa, Artificial sequence
  N-terminal antigen binding fragment (Fab) of SYNAGIS heavy chain humanized antibody
QVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTTCTCSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTH

SEQ ID NO: 3 - protein, 7 aa, Artificial sequence
  Complementary determining region (CDR) H1 of SYNAGIS heavy chain humanized antibody
TSGMSVG

SEQ ID NO: 4 - protein, 16 aa, Artificial sequence
  Complementary determining region (CDR) H2 of SYNAGIS heavy chain humanized antibody
DIWWDDKKDYNPSLKS

SEQ ID NO: 5 - protein, 10 aa, Artificial sequence
  Complementary determining region (CDR) H3 of SYNAGIS heavy chain humanized antibody
SMITNWYFDV

SEQ ID NO: 6 - protein, 213 aa, Artificial sequence
  Light chain of SYNAGIS humanized antibody
DIQMTQSPSTLSASVGDRVTITCKCQLSVGYMHWYQQKPGKAPKLLIYDTSKLASGVPSRFSGSGSGTEFTLTISSLQPDDFATYYCFQGSGYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 10 aa, Artificial sequence
  Complementary determining region (CDR) L1 of SYNAGIS light chain humanized antibody
KCQLSVGYMH

SEQ ID NO: 8 - protein, 7 aa, Artificial sequence
  Complementary determining region (CDR) L2 of SYNAGIS light chain humanized antibody
DTSKLAS

SEQ ID NO: 9 - protein, 9 aa, Artificial sequence
  Complementary determining region (CDR) L3 of SYNAGIS light chain humanized antibody
FQGSGYPFT